(19) United States
(12) Patent Application Publication
(10) Pub. No.: US 2009/0169475 A1
(43) Pub. Date: Jul. 2, 2009

(54) RADIOLABELED DERIVATIVES OF POTENT CHYMASE INHIBITORS

(75) Inventors: John W. Babich, Cambridge, MA (US); William C. Eckelman, Cambridge, MA (US); John Joyal, Melrose, MA (US); Craig Zimmerman, Topsfield, MA (US)

Correspondence Address: FOLEY AND LARDNER LLP;SUITE 500, 3000 K STREET NW, WASHINGTON, DC, 20007, US

(73) Assignee: MOLECULAR INSIGHT PHARMACEUTICALS, INC.

(21) Appl. No.: 12/338,426

(22) Filed: Dec. 18, 2008

Related U.S. Application Data
(60) Provisional application No. 61/015,977, filed on Dec. 21, 2007.
(60) Provisional application No. 61/019,456, filed on Jan. 7, 2008.

(51) Int. Cl. A61K 51/00; C07D 333/52; C07F 9/28

(52) U.S. Cl. 424/1.89; 549/6; 548/412; 424/1.85

(57) ABSTRACT

Methods of imaging tissue of a mammal which expresses chymase include administering to the mammal an effective amount of a radiolabeled chymase inhibitor. Radiopharmaceuticals that may be used in diagnostic imaging and therapeutic treatment of disease characterized by expression of chymase have the structure of Formula I:

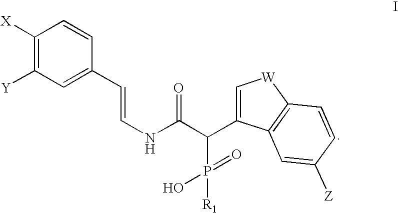

### CROSS-REFERENCE TO RELATED APPLICATIONS

This application claims the benefit of U.S. Provisional Patent Application Nos. 61/019,456, filed Jan. 7, 2008, and 61/015,977, filed Dec. 21, 2007, the entire contents of which are incorporated herein by reference for any and all purposes.

### FIELD

This invention relates in general to radiopharmaceuticals for diagnostic imaging and therapeutic treatment of diseases, and in particular, to radioactive halogenated derivatives of potent chymase inhibitors useful in diagnostic imaging and treatment of diseases including heart disease and congestive heart failure.

### BACKGROUND

It is estimated that approximately 5 million people in the United States have congestive heart failure (CHF). About 550 thousand new cases are diagnosed each year. More than 287,000 patients in the United States die each year from CHF. Hospitalizations for CHF have increased substantially. Admissions rose from 402,000 in 1979 to 1,101,000 in 2004. Heart failure is the most common reason for hospitalization among Medicare patients. From 1993-2003, deaths from heart failure increased 20.5%. In the same time period, the overall death rate declined 2%. The 2003 overall death rate for heart failure was 19.7 per 100,000. The estimated direct cost for heart failure in 2006 is $29.6 billion in the United States.

The most common causes of congestive heart failure are coronary artery disease, hypertension or high blood pressure, and diabetes. Angiotensin-converting enzyme (ACE) inhibitors have been assessed in millions of patients and widely used in preventing morbidity and mortality in patients with hypertension, left ventricular dysfunction, heart failure, and atherosclerotic disease. Angiotensin-converting enzyme is a transmembrane dipeptidyl peptidase enzyme that catalyzes the conversion of angiotensin I (ANG I) to angiotensin II (ANG II), which is a potent vasoconstrictor. However, the use of ACE inhibitors in treating congestive heart failure has not been uniformly successful. Studies have suggested that full suppression of the rennin-angiotensin system (RAS) cannot be achieved by ACE inhibition alone. There is emerging evidence that not all patients need or benefit from ACE inhibitors. Other drugs such as diuretics, beta-adrenergic blockers, calcium channel blockers, and drug classes that inhibit portions of the rennin-angiotensin system have shown effects in some patients, indicating that other sources of angiotensin II, such as chymase conversion of angiotensin I to angiotensin II, may be important. Chymase (EC 3.4.21.39) is a chymotrypsin-like enzyme that is expressed in the secretory granule of mast cells. Therefore, it is important to evaluate both ACE inhibitors and chymase inhibitors in CHF patients, including their combinations.

Medical imaging technology plays a significant role in diagnosis and treatment of diseases. For instance, computed tomography (CT) is used to provide anatomic information of a patient by generating cross-sectional images using X-ray transmission. Positron emission tomography (PET), single photon emission computed tomography (SPECT) and scintigraphy are used to target specific tissues or organs to provide pharmacological information about a patient. In PET, SPECT, and scintigraphy, radiopharmaceuticals capable of sequestering, to some degree, in the target tissue or organ, are internally administered to a patient, and images are generated by detecting the radioactive emissions from the sequestered radiopharmaceuticals. Radiopharmaceuticals include radionuclides which produce X-ray or gamma-ray emissions or positrons as they decay.

The amount and type of clinical information that can be derived from PET, SPECT, and scintigraphic images is related, in part, to the ability of the radiopharmaceuticals to sequester in the target tissue or organ. Radiopharmaceuticals have been used in a variety of types of studies to obtain different kinds of information. For example, radiopharmaceuticals used in cardiac blood flow and blood pool studies provide information on murmurs, cyanotic heart disease, and ischemic heart disease. Perfusion scintigraphy agents provide measurements of blood flow useful in detection of coronary artery disease, assessment of pathology after coronary arteriography, pre- and postoperative assessment of coronary artery disease, and detection of acute myocardial infarction. Infarct avid agents are used for “hot spot” infarct imaging. Radiopharmaceuticals which allow binding to specific cardiac receptors may allow detection of highly specific binding in the cardiovascular system. Radiopharmaceutical agents capable of detecting the rate and amount of metabolism are particularly important to the progress of clinical nuclear medicine, since they allow studies of the energy consumption in the various stages of disease processes.

Previous studies on the ACE inhibitors and chymase inhibitors contain inconsistencies regarding the effectiveness of either inhibitor in heart diseases. For example, a study by Jin et al. (Impact Of Chymase Inhibitor On Cardiac Function And Survival After Myocardial Infarction, Cardiovascular Research 2003:60: 413-420.) suggests that the increase in ANG II production via activated cardiac chymase plays an important role after myocardial infarction (MI). According to Jin et al., the different effects of ACE inhibitors in rat and hamster following myocardial infarction depend on whether or not the cardiac tissues contain ANG II-generating chymase. In a study by Akasu et al. (Differences In Tissue Angiotension II-Forming Pathways By Species And Organs In Vitro, Hypertension 1998:32:514-520.), it is concluded that the enzyme that is responsible for pulmonary ANG II formation is ACE in all of the studied species except the human lung, in which a chymase like enzyme is dominant. A study by De Lannoy et al. (Angiotensin Converting Enzyme Is The Main Contributor To Angiotensin I-II Conversion In The Interstitium Of The Isolated Perfused Rat Heart, J Hypertens 1999:19:959-965) using isolated perfused rat heart suggests that ACE is responsible for production of ANG II, although the conclusion has not been substantiated by performing ANG I perfusion experiments in the presence of a selective chymase inhibitor. According to De Lannoy et al., the accessibility of chymase may be different under pathological conditions, e.g., after myocardial infarction. Since chymase may not be as readily available in intact hearts, an in vivo model needs to be pursued in subsequent studies.

The availability of high affinity, specific chymase inhibitors complementary to high affinity, specific ACE inhibitors will help determine whether it is the animal model or species differences that are responsible for the inconsistent conclusions in the studies of hear diseases. Radioactive ACE inhibitors have been developed for positron emission tomography. For example, ¹⁸F fluorocaptopril and ¹⁸F fluorobenzoyllisinopril have been recently developed by Molecular Insight Pharmaceuticals, Inc. (MIP) and are moving their way toward clinical studies. There are needs for radioactive chymase inhibitors so that the roles of angiotensin-converting enzyme and chymase can be studied and compared in both animal models and humans using nuclear medicine imaging technology such as positron emission tomography and single photon emission computed tomography.

### SUMMARY

Radiopharmaceuticals are provided that are useful in diagnostic imaging and therapeutic treatment of a disease that is characterized by expression of chymase such as hypertension, diabetes, left ventricular dysfunction, heart failure, and atherosclerotic disease. The radiopharmaceuticals include a benzothiophene phosphonic acid derivative or indole phosphinic acid derivative that affords excellent affinity for chymase, and a radioactive halogen that provides radiotracers for PET or SPECT imaging.

In one aspect, a compound of Formula I, its stereoisomer or pharmaceutically acceptable salt is provided:

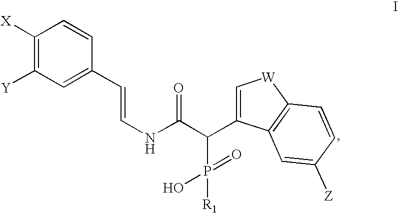

where R₁ is hydroxyl or an alkyl group having 1 to 6 carbon atoms; W is a sulfur atom or N—R₂ group where R₂ is hydrogen or an alkyl group having 1 to 6 carbon atoms; X is hydrogen, or a radioactive or non-radioactive halogen; Y is a radioactive or non-radioactive halogen; Z is a radioactive or non-radioactive halogen; and one of X, Y, and Z is a radioactive halogen.

In some preferred embodiments, the radioactive halogen is selected from the group consisting of ¹⁸F, ¹²³I, ¹²¹I, ¹³¹I, and ⁷⁶Br.

In some preferred embodiments, W of Formula I is a sulfur atom or N-methyl group, and R₁ is hydroxyl or methyl group.

In some preferred embodiments, compounds having formulas I-a to I-d are provided:

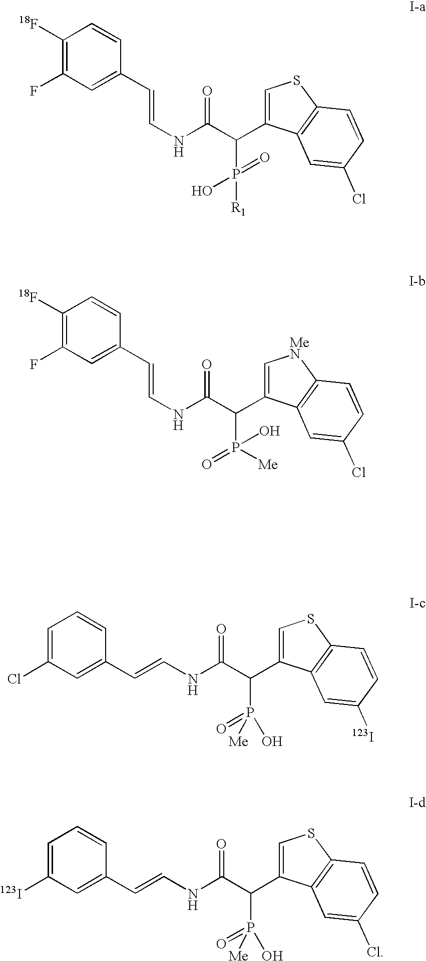

In one aspect, a method of imaging tissue of a mammal which expresses chymase is provided which comprises administering to the mammal an effective amount of a radiolabeled compound that selectively inhibits chymase. In a preferred embodiment of the invention, the radiolabeled compound includes a radioactive halogenated derivative of a potent chymase inhibitor. In a particularly preferred embodiment, an effective amount of a compound of Formula I, its stereoisomer or pharmaceutically acceptable salt is administered to the mammal.

In a further aspect, a method of inhibiting the enzymatic activity of chymase in a mammal or treating a mammal suffering from a disease characterized by overexpression of chymase is provided which comprises administering to said mammal an effective amount of a radiolabeled chymase inhibitor, preferably a radioactive halogen derivative, and more preferably a compound of Formula I, its stereoisomer or pharmaceutically acceptable salt.

### DETAILED DESCRIPTION

Various embodiments of the invention are described hereinafter. It should be noted that the specific embodiments are not intended as an exhaustive description of the invention or as a limitation on the scope of the invention. One aspect described in conjunction with a particular embodiment of the present invention is not necessarily limited to that embodiment and can be practiced with any other embodiment(s) of the invention.

As used herein, the following definitions of terms shall apply unless otherwise indicated.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The embodiments, illustratively described herein may suitably be practiced in the absence of any element or elements, limitation or limitations, not specifically disclosed herein. Thus, for example, the terms “including,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention. Additionally the phrase “consisting essentially of” will be understood to include those elements specifically recited and those additional elements that do not materially affect the basic and novel characteristics of the claimed invention. The phrase “consisting of” excludes any element not specifically specified.

Radiolabeled chymase inhibitors are provided for use in diagnostic imaging and treatment of diseases which are characterized by expression of chymase such as hypertension, diabetes, left ventricular dysfunction, heart failure, and atherosclerotic disease. The radiolabeled chymase inhibitors can be any radiolabeled compound that selectively inhibits chymase. Accordingly, the identification of compounds that afford affinity for chymase and contain a halogen that is amenable to replacement with a radioactive halogen, are provided. In some embodiments, the compounds are benzothiophene phosphonic acid derivatives or indole phosphinic acid derivatives that contain an aromatic halide and afford excellent affinity for chymase. Replacement of the halogen in the identified compounds with a radionuclide by radiohalogenation such as radiofluorination or radioiodination provides compounds that both afford affinity for chymase and provide radiotracers for PET or SPECT imaging.

Accordingly, in one aspect, provided is a compound of Formula I, its stereoisomer or pharmaceutically acceptable salt:

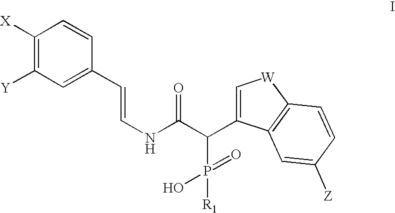

wherein R₁ is hydroxyl or an alkyl group having 1 to 6 carbon atoms; W is a sulfur atom or N—R₂ group where R₂ is hydrogen or an alkyl group having 1 to 6 carbon atoms; X is hydrogen, a radioactive or non-radioactive halogen; Y is a radioactive or non-radioactive halogen; Z is a radioactive or non-radioactive halogen; and one of X, Y, and Z is a radioactive halogen. As used herein, the term “radioactive halogen” in Formula I refers to ¹⁸F, ¹²³I, ¹²⁵I, ¹³¹I, or ⁷⁶Br.

As used herein, the phrase “alkyl group having 1 to 6 carbon atoms” refers to monovalent saturated aliphatic hydrocarbyl groups having 1 to 6 carbon atoms. The phrase includes, by way of example, linear and branched hydrocarbyl groups such as methyl, ethyl, n-propyl, isopropyl, n-butyl, isobutyl, sec-butyl, t-butyl, n-pentyl, iso-pentyl, neopentyl, 2,2-dimethylpropyl, n-hexyl, and, as well as branched isomeric forms of hexyl.

In some embodiments, W a sulfur atom, and R₁ is hydroxyl or an alkyl group having 1-6 carbon atoms, and at least one of X, Y and Z is a radioactive halogen. In some embodiments, W is a sulfur atom, R₁ is hydroxyl, X is a radioactive halogen, and Y and Z is a non-radioactive halogen. In other embodiments, W is a sulfur atom, R₁ is hydroxyl group, X is a ¹⁸F, and Y and Z are independently a non-radioactive halogen. In other embodiments, W is a sulfur atom, R₁ is methyl group, and one of X, Y, and Z is a radioactive halogen.

In some embodiments, W is a sulfur atom, R₁ is methyl group, and X is hydrogen. In such embodiments, Y can be a radioactive halogen, and Z a non-radioactive halogen. Alternatively, Y may be a non-radioactive halogen, and Z a radioactive halogen. For example, in some embodiments, W is a sulfur atom, R₁ is methyl group, X is hydrogen, Y is a non-radioactive halogen, and Z is ¹²³I. In other embodiments, W is a sulfur atom, R₁ is methyl group, X is hydrogen, Y is ¹²³I, and Z is a non-radioactive halogen.

In some embodiments, W is an N—R₂ group where R₂ is an alkyl group having 1-6 carbon atoms, and R₁ is hydroxyl or an alkyl group having 1 to 6 carbon atoms. In other embodiments, W is N-methyl group and R₁ is methyl group. In other embodiments, W is N-methyl group and R₁ is methyl group, X is a radioactive halogen, and Y and Z are independently a non-radioactive halogen. In other embodiments, W is N-methyl group and R₁ is methyl group, X is ¹⁸F, and Y and Z are independently a non-radioactive halogen.

One skilled in the art will readily realize that all ranges discussed can and do necessarily also describe all subranges therein for all purposes and that all such subranges also form part and parcel of this invention. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

Table 1 provides some exemplary compounds having the structure of the compound of Formula I. It should be noted that the compounds are provided for illustrative purpose and are not intended to limit the scope of the invention.

TABLE 1
Exemplary Compounds of Formula I.

| | |
|---|---|
| | I |
| 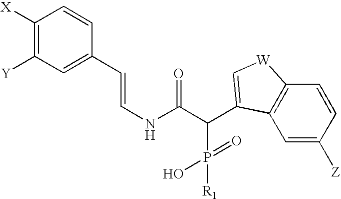 | |

| Compound | | | | | |
|---|---|---|---|---|---|
| No. | W | R₁ | X | Y | Z |
| 1 | S | OH | ¹⁸F | F | Cl |
| 2 | S | OH | F | F | ¹²³I |
| 3 | S | OH | H | H | ¹²³I |
| 4 | S | Me | ¹⁸F | F | Cl |
| 5 | S | Me | F | F | ¹²³I |
| 6 | S | Me | H | ¹²³I | Cl |
| 7 | S | Me | ¹²³I | H | Cl |
| 8 | N-Me | OH | ¹⁸F | F | Cl |
| 9 | N-Me | OH | F | F | ¹²³I |
| 10 | N-Me | OH | H | H | ¹²³I |
| 11 | N-Me | Me | ¹⁸F | F | Cl |
| 12 | N-Me | Me | F | F | ¹²³I |
| 13 | N-Me | Me | H | H | ¹²³I |
| 14 | N-Me | Me | ¹²³I | H | Cl |
| 15 | N-Me | Me | H | ¹²³I | Cl |

In some embodiments, ¹⁸F-containing or ¹²³I-containing benzothiophene or N-alkylindole phosphonic and phosphinic acids derivatives have Formulas I-a, I-b, I-c, or I-d:

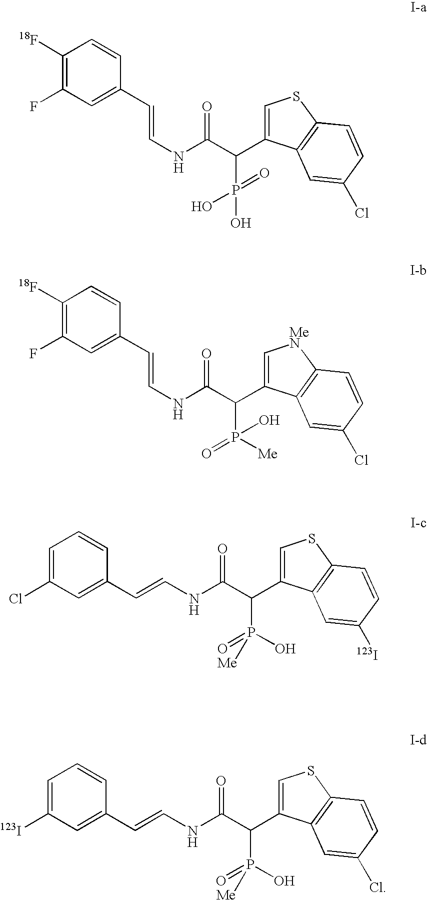

The compounds represented by Formula I may be prepared using any of the synthetic schemes set forth below. In general, the compound represented by Formula I may be prepared by radiofluorination or radioiodination of a radiolabeling precursor using commercially available radioactive fluorine or iodine. The radiolabeling precursor can be alkylammonium triflate salts of benzothiophene phosphonate derivative or indole phosphinate derivative, which can be prepared from isocyanate derivatives by condensations with corresponding benzothiophene or indole derivatives.

### Synthesis of Benzothiophene Derivatives Containing ¹⁸F

The synthesis of benzothiophene phosphonic acid derivatives containing ¹⁸F (I-a) may begin with the commercially available benzaldehyde (11). Wittig reaction of the benzaldehyde (11) with the ylide methyl(triphenylphosphoranylidene) acetate affords the cinnamate (12), which is converted into the isocyanate (13) after hydrolysis of the methyl ester and treatment with diphenylphosphoryl azide.

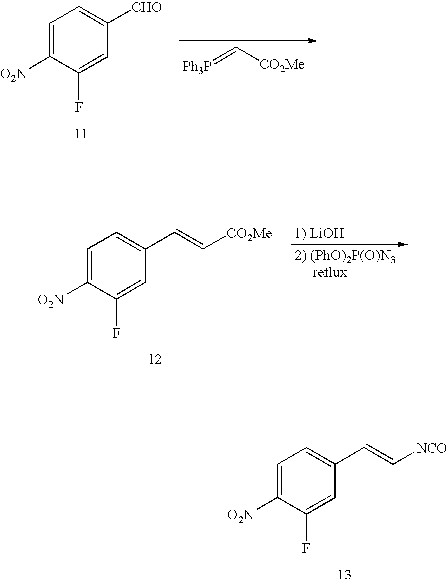

Commercially available (bromomethyl)-benzothiophene (14) is converted into phosphonate (15) by treatment with triethylphosphite.

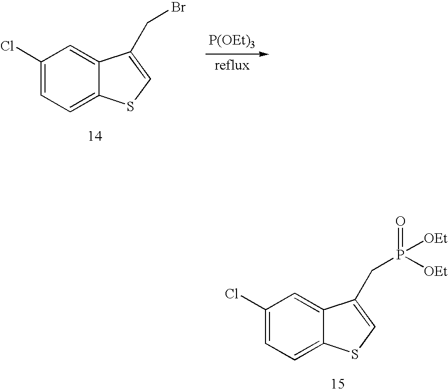

Deprotonation of phosphonate (15) with butyllithium followed by condensation with the isocyanate (13) affords benzothiophene phosphonate derivative (16).

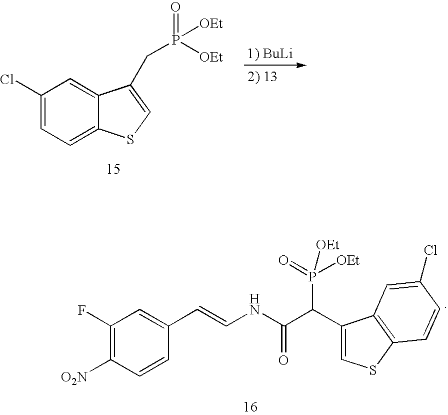

Benzothiophene phosphonate (16) is then converted to an aniline derivative by reduction, after which treatment with excess methyl triflate yields the radiolabeling precursor, the trimethylammonium triflate salt (17). The introduction of the ¹⁸F atom into the 4-position of the electron poor aryl ring is assisted by the electron poor 3-fluoro group. The radiofluorination of (17) is accomplished under standard conditions utilizing [¹⁸F]-KF in the presence of the phase transfer catalyst Kryptofix. Benzothiophene phosphonic acid (I-a) is obtained after rapid de-ethylation with trimethylsilyl bromide (TMS-Br). Alternatively, the radiofluorination can be conducted after de-ethylation of (17). Therefore, treatment of (17) with TMS-Br to afford the phosphonic acid followed by treatment with [F-18]-KF in the presence of Kryptofix can also result in the desired benzothiophene phosphonic acid (I-a).

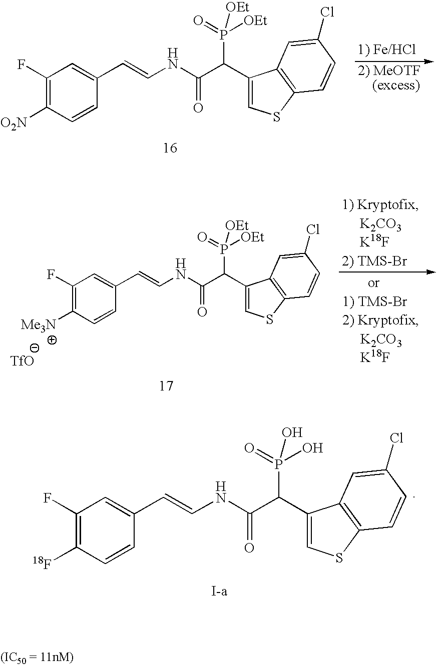

### Synthesis of Indole Derivatives Containing ¹⁸F

The synthesis of indole phosphinic acid derivatives containing ¹⁸F (I-b) may begin with commercially available chloroindole (18). Reaction of chloroindole (18) with Eschenmoser's salt affords (19) which after methylation to the trimethylammonium iodide salt is reacted with diethylmethylphophite to afford the indole phosphonate (20). Methylation of the indole phosphinate (20) with sodium hydride and methyl iodide affords N-methylindole phosphinate (21).

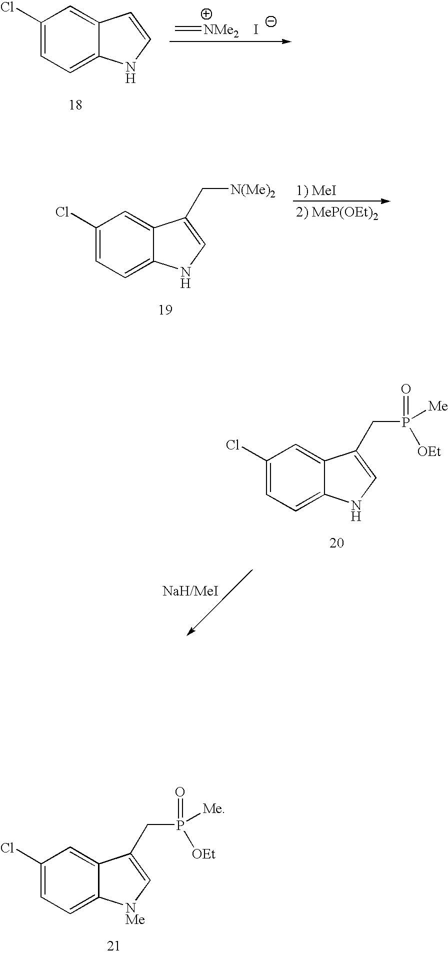

Deprotonation of N-methylindole phosphinate (21) followed by condensation with the isocyanate (22) affords the nitro derivative (23):

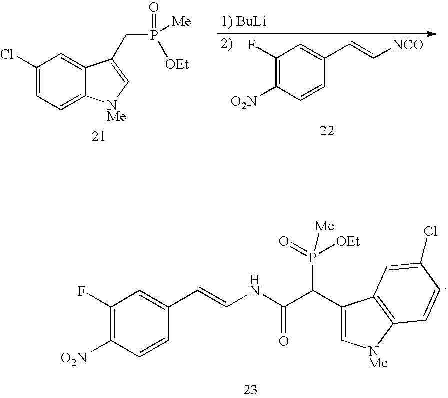

Reduction of the nitro derivative (23) to an aniline derivative followed by reaction with methyl triflate affords the radiolabeling precursor, the trimethylammonium triflate salt (24). The radiofluorination of the radiolabeling precursor (24) followed by de-ethylation yields the desired N-methylindole phosphinic acid derivative (I-b). The radiofluorination can also be conducted after the de-ethylation of (24). Therefore, treatment of (24) with trimethylsilyl bromide (TMS-Br) to afford the phosphinic acid followed by treatment with [¹⁸F]-KF in the presence of Kryptofix can also result in the desired N-methylindole phosphinic acid derivative (I-b).

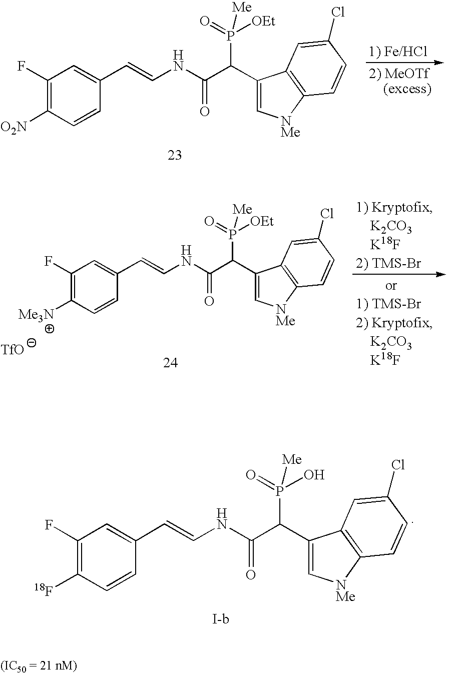

### Synthesis of Benzothiophene Derivatives Containing ¹²³I

The synthesis of ¹²³I-containing benzothiophene phosphinic acid derivatives is similar to that of ¹⁸F-containing benzothiophene phosphinic acid derivatives described above although the introduction of the iodine is by an electrophilic reaction rather than by a nucleophilic reaction. The iodobenzothiophene (28) is converted into the methylphosphinate (29) by treatment with diethylmethylphosphite. Deprotonation with sodium hydride followed by quenching with the isocyanate (30) gives (31), which can be converted into the trimethylstannane radiolabeling precursor (32) by the treatment with hexamethyldistannane under palladium catalysis. Radioiodination of trimethylstannane radiolabeling precursor (32) under standard conditions using [¹²³I]—NaI followed by rapid de-ethylation with TMS-Br affords the ¹²³I-containing benzothiophene phosphinic acid derivative (I-c).

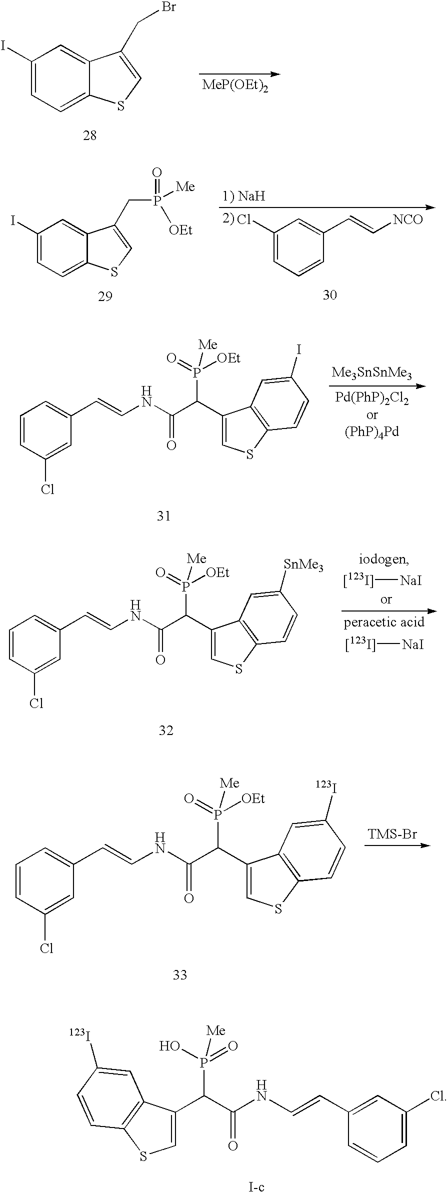

### Synthesis of Benzothiophene Derivatives Containing ¹²³I

The synthesis of ¹²³I-containing benzothiophene phosphinic acid derivatives (I-d) proceeds through a similar route to (I-c). Deprotonation of the benzothiophene phosphinate (34) with BuLi followed by reaction with isocyanate (35) affords (36). Treatment of (36) with hexamethyldistannane under palladium catalysis affords the radiolabeling precursor trimethylstannane (37). Radioiodination of the trimethylstannane (37) under standard conditions using [¹²³I]—NaI affords (38), which after rapid de-ethylation with TMS-Br affords the ¹²³I-containing benzothiophene phosphinic acid derivative (I-d).

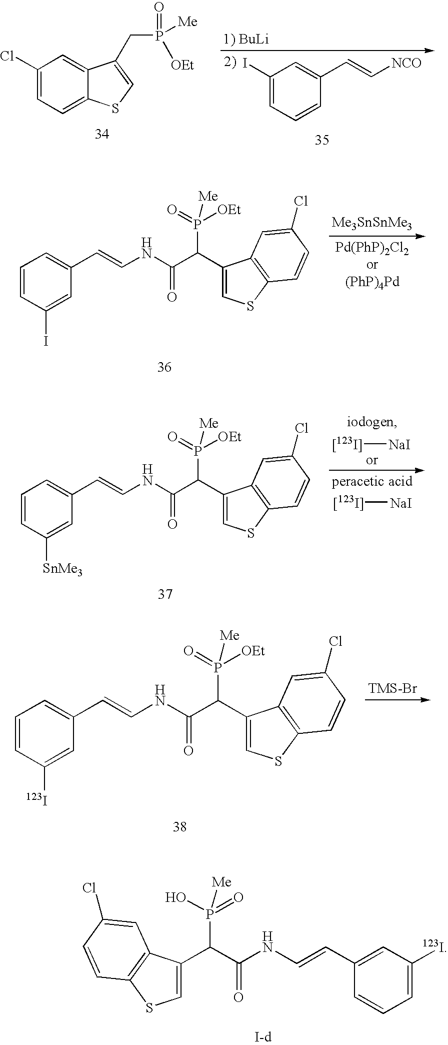

The radioactive halogenated chymase inhibitors can be used as imaging agents in accordance with methods known to those skilled in the art. An effective amount of the imaging agent (e.g., from 1 to 50 mCi) is combined with a pharmaceutically acceptable carrier for use in imaging studies. As used herein, “an effective amount of the imaging agent” is defined as an amount sufficient to yield an acceptable image using equipment which is available for clinical use. An effective amount of the imaging agent may be administered in more than one injection. Effective amounts of the imaging agent vary according to factors such as the degree of susceptibility of the individual, the age, sex, and weight of the individual, idiosyncratic responses of the individual and dosimetry. The effective amount of the imaging agent will vary according to instrument and film-related factors. Optimization of such factors is well within the level of skill in the art.

As used herein, “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. The radioactive halogenated chymase inhibitors of the invention may further be administered to an individual in an appropriate diluent or adjuvant, co-administered with other enzyme inhibitors such ACE inhibitors or in an appropriate carrier such as human serum albumin or liposomes. Pharmaceutically acceptable diluents include saline and aqueous buffer solutions. Adjuvants contemplated herein include resorcinols, non-ionic surfactants such as polyoxyethylene oleyl ether and n-hexadecyl polyethylene ether.

The radioactive halogenated chymase inhibitors may be used as a therapeutic agent in accordance with methods known to those skilled in the art. A therapeutically effective amount of the chymase inhibitor is combined with a pharmaceutically acceptable carrier and administered to a patient suffering from a disease such as congestive heart failure. As used herein, the term “therapeutically effective amount” refers to a therapeutically effective, chymase inhibitive amount of a compound of Formula I. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to: the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the use of concomitant medication; and other relevant circumstances.

Preferably, the radiopharmaceuticals are administered intravenously, and may be formulated as a sterile, pyrogen-free, parenterally acceptable aqueous solution. The preparation of such parenterally acceptable solutions, having due regard to pH, isotonicity, stability, and the like, is within the skill in the art. In one embodiment, formulation for injection contains, in addition to the cardiovascular imaging agent, an isotonic vehicle such as Sodium Chloride Injection, Ringer's Injection, Dextrose Injection, Dextrose and Sodium Chloride Injection, Lactated Ringer's Injection, or other vehicle as known in the art. The formulation used in the may also contain stabilizers, preservatives, buffers, antioxidants, or other additives known to those of skill in the art.

### Measurement of Enzyme Activity

Chymase activity can be measured according to the method described in Cooley J, et al., Biochemistry 2001, 40, 15762-15770. Briefly, purified human skin chymase (Biomol) is incubated with test compounds and substrate (succinyl-Ala-Ala-Pro-Phe-p-nitroanilide) (Biomol) in 200 mM Tris-HCl, pH 8.0, 1 M NaCl at 25° C. Cleavage of the substrate is monitored at 410 nm.

Angiotensin-converting enzyme (ACE) activity can be measured according to the method described by Femia et al., in: Mazzi U, Giron, M C, eds. Technetium, Rhenium and Other Metals in Chemistry and Nuclear Medicine 7. Padova, Italy: Servizi Grafici Editoriali; 2006:627-630. Briefly, purified rabbit lung ACE (Sigma Chemicals) is incubated with test compound and substrate (p-hydroxybenzoyl-glycyl-L-histidyl-L-leucine) at 37° C. Developer solution, which promotes a color change, is added and the samples are incubated for an additional 5 minutes at 37° C. before reading at 505 nm.

### Rat and Hamster Tissue Distribution Studies

Tissue distribution studies of radiohalogenated chymase inhibitors can be performed in separate groups of male Sprague-Dawley rats (n=5/time point) and male Syrian hamsters (n=5/time point) administered as a bolus intravenous injection (approximately 10 μCi/rat) in a constant volume of 0.1 ml and 2 μCi/hamster in a constant volume of 0.05 ml.

To examine specificity, separate groups of rats and hamsters are injected intravenously with non-radiolabeled chymase inhibitor five minutes prior to injection of the radiolabeled chymase inhibitor. Groups of animals are euthanized by asphyxiation with carbon dioxide at 30 minutes, 60 min, or 120 post injection. Blood, heart, lungs, liver, spleen, kidneys, large and small intestines (with contents), testes, skeletal muscle, and adipose are excised, weighed wet, transferred to plastic tubes and counted in an automated g-counter (LKB Model 1282, Wallac Oy, Finland). Aliquots of the injected dose are measured to convert the counts per minute in each tissue sample to percent-injected dose per organ. Tissue radioactivity levels of the radioactivity expressed as % ID/g are determined by converting the decay corrected counts per minute to the percent dose and dividing by the weight of the tissue or organ sample. Chymase specific uptake is judged based on the rank order of tissue uptake as compared to literature values of chymase expression determined by immunohistochemistry, and the extent of pharmacological blocking using the cold chymase inhibitor as well as structurally unrelated chymase inhibitors.

| Radiotracer | Sham injection | Cold Chymase inhibitor |
|---|---|---|
| Chymase inhibitor | 15 rats, 15 hamsters | 15 rats, 15 hamsters |

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A compound of Formula I, its stereoisomer or pharmaceutically acceptable salt: 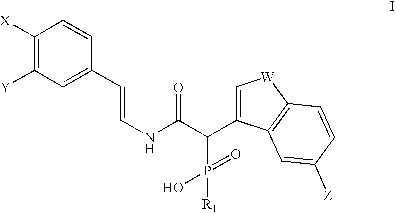
wherein:
R₁ is hydroxyl or an alkyl having 1 to 6 carbon atoms;
W is a sulfur atom or N—R₂ group;
R₂ is hydrogen or an alkyl group having 1 to 6 carbon atoms;
X is hydrogen, or a radioactive or non-radioactive halogen;
Y is a radioactive or non-radioactive halogen;
Z is a radioactive or non-radioactive halogen; and
one of X, Y, and Z is a radioactive halogen.

2. The compound of claim 1, wherein said radioactive halogen is ¹⁸F, ¹²³I, ¹²⁵I, ¹³¹I, or ⁷⁶Br.

3. The compound of claim 1, wherein W is a sulfur atom, R₁ is hydroxyl group or an alkyl group having 1 to 6 carbon atoms, and one of X, Y and Z is a radioactive halogen.

4. The compound of claim 1, wherein W is a sulfur atom, R₁ is hydroxyl group, X is a radioactive halogen, and Y and Z are independently a non-radioactive halogen.

5. The compound of claim 1, wherein W is a sulfur atom, R₁ is hydroxyl group, X is ¹⁸F, and Y and Z are independently a non-radioactive halogen.

6. The compound of claim 1, wherein W is a sulfur atom, and R₁ is methyl group.

7. The compound of claim 1, wherein W is a sulfur atom, R₁ is methyl group, Z is ¹²³I, X is hydrogen, and Y are independently a non-radioactive halogen.

8. The compound of claim 1, wherein W is a sulfur atom, R₁ is methyl group, Y is ¹²³I, X is hydrogen, and Z is a non-radioactive halogen.

9. The compound of claim 1, wherein W is a N—R₂ group, R₂ is an alkyl group having 1 to 6 carbon atoms, and R₁ is an alkyl group having 1 to 6 carbon atoms.

10. The compound of claim 1, wherein W is an N-methyl group, R₁ is methyl group, X is ¹⁸F, and Y and Z are independently a non-radioactive halogen.

11. The compound of claim 1 which is:

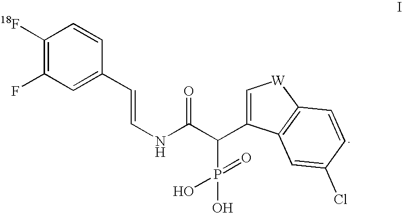

12. The compound of claim 1 which is:

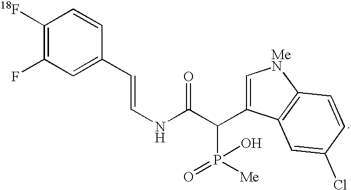

13. The compound of claim 1 which is:

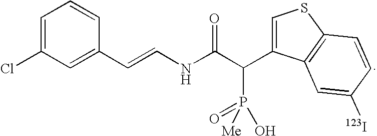

14. The compound of claim 1 which is:

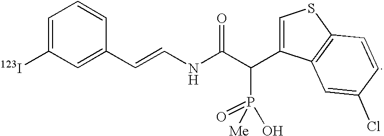

15. A method of imaging tissue of a mammal which expresses chymase comprising administering to said mammal an effective amount of a compound of Formula I, its stereoisomer or pharmaceutically acceptable salt: 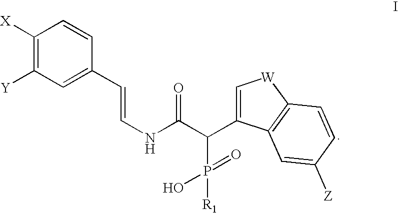
wherein:
R₁ is hydroxyl or an alkyl having 1 to 6 carbon atoms;
W is a sulfur atom or N—R₂ group;
R₂ is hydrogen or an alkyl group having 1 to 6 carbon atoms;
X is hydrogen, or a radioactive or non-radioactive halogen;
Y is a radioactive or non-radioactive halogen;
Z is a radioactive or non-radioactive halogen; and
one of X, Y, and Z is a radioactive halogen.

16. The method of claim 15, wherein said radioactive halogen is ¹⁸F, ¹²³I, ¹²⁵I, ¹³¹I, or ⁷⁶Br.

17. The method of claim 15, wherein W is a sulfur atom or N-methyl group, and R₁ is hydroxyl or methyl group.

18. The method of claim 15, wherein the compound is:

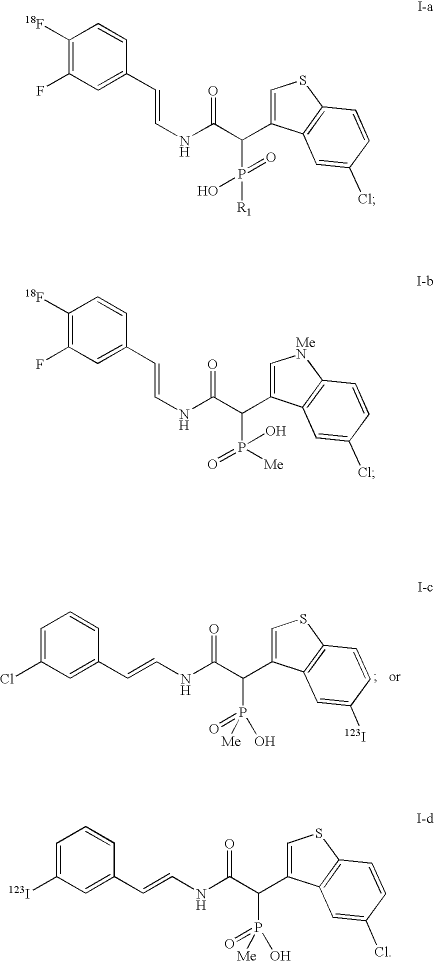

19. The method of claim 15, wherein said mammal suffers from hypertension, diabetes, left ventricular dysfunction, heart failure, or atherosclerosis.

20. The method of claim 19 further comprising administering to said mammal an effective amount of angiotensin-converting enzyme inhibitor.

21. A method of imaging tissue of a mammal which expresses chymase comprising administering to said mammal an effective amount of a radiolabeled chymase inhibitor.

22. The method of claim 21, wherein said mammal suffers from a disease of hypertension, diabetes, left ventricular dysfunction, heart failure, or atherosclerosis.

23. The method of claim 22 further comprising administering to said mammal an effective amount of a radiolabeled angiotensin-converting enzyme inhibitor.
